(19) United States
(12) Patent Application Publication
(10) Pub. No.: US 2025/0340856 A1
(43) Pub. Date: Nov. 6, 2025

(54) Minimal Peptide Fusions for Targeted Intracellular Degradation of FOXP3

(71) Applicant: Massachusetts Institute of Technology, Cambridge, MA (US)

(72) Inventors: Pranam Chatterjee, Durham, NC (US); Joseph M. Jacobson, Newton, MA (US)

(73) Assignee: Massachusetts Institute of Technology, Cambridge, MA (US)

(21) Appl. No.: 19/078,044

(22) Filed: Mar. 12, 2025

Related U.S. Application Data
(62) Division of application No. 17/972,132, filed on Oct. 24, 2022, now abandoned.
(60) Provisional application No. 63/271,024, filed on Oct. 22, 2021.

(51) Int. Cl. C12N 9/48

(52) U.S. Cl. CPC C12N 9/48; C12Y 304/17023

(57) ABSTRACT

Peptide-E3 ubiquitin ligase fusions representing minimal protein to proteasome linkers are specifically targeted to degrade endogenous FOXP3 proteins in regulatory T cells. An engineered peptide for functional inactivation of a target regulatory T cell includes a fusion protein comprising a targeting domain and a ubiquitin ligase recruiting domain, wherein the targeting domain is engineered to bind FOXP3 of the target regulatory T cell for mediated degradation by the ubiquitin-proteosome pathway. The targeting domain may comprise a peptide having amino acid [SEQ ID No. 3], [SEQ ID No. 4], [SEQ ID No. 5], or [SEQ ID No. 6]. The ubiquitin ligase recruiting domain recruits an E3 ubiquitin ligase, which may be CHIPΔR [SEQ ID No. 2]. An engineered minimal, specific, nucleotide-encodable, FOXP3 protein to proteasome linker comprises a peptide-E3 ubiquitin ligase fusion in which the peptide binds to FOXP3. A method for treatment includes administering to a subject an engineered peptide-based therapeutic or pharmaceutically acceptable salt thereof, wherein the engineered peptide-based therapeutic comprises a peptide fusion of a targeting domain and a ubiquitin ligase recruiting domain, and wherein the targeting domain is engineered to bind FOXP3 of at least one regulatory T cell for mediated degradation by the ubiquitin-proteosome pathway.

### RELATED APPLICATIONS

This application is a divisional of U.S. patent application Ser. No. 17/972,132, filed Oct. 24, 2022, which claims the benefit of U.S. Provisional Application Ser. No. 63/271,024, filed Oct. 22, 2021, the entire disclosures of which are herein incorporated by reference in their entirety.

### INCORPORATION BY REFERENCE OF SEQUENCE LISTING XML

This application contains a Sequence Listing XML submitted under the provisions of 37 CFR 1.831 (a) and herein incorporated by reference. The Sequence Listing XML includes, in XML format, the following file:

| File name | Creation Date | Size in bytes |
|---|---|---|
| MIT1250.xml | Mar. 7, 2023 1:08 PM | 8000 |

### FIELD OF THE TECHNOLOGY

The present invention relates to peptides for targeting regulatory T cells for intracellular degradation, and in particular to minimal fusion peptides with an ubiquitin ligase and a targeting domain binding to a FOXP3 protein receptor.

### BACKGROUND

Targeted protein depletion is a key method of disrupting protein-protein interactions and protein function in vivo. Protein synthesis can be blocked at various levels. At the DNA level, protein coding genes can be disrupted using genome editing tools, such as zinc-finger nucleases, TALENs, and CRISPR-Cas9 [Gaj, et al., “ZFN, TALEN, and CRISPR/Cas-based methods for genome engineering”, Trends Biotechnol. (2013)]. At the post-transcriptional level, methods such as RNAi or CRISPR-Cas13 can be used for degrading targeted messenger RNAs (mRNAs) [Boettcher, et al., “Choosing the right tool for the job: RNAi, TALEN or CRISPR”, Mol. Cell (2015)]. Finally, at the translational level, antisense oligonucleotides can be utilized to hybridize to the mRNA and block the progression of the translation initiation complex from the 5′ cap to the start codon [Eisen, J. S. et al., “Controlling morpholino experiments: don't stop making antisense”, Development (2008)].

The most rapid and acute method of protein degradation intracellularly, however, is at the post-translational level. Specifically, E3 ubiquitin ligases can tag endogenous proteins for subsequent degradation in the proteasome [Ardley, et al., “E3 ubiquitin ligases”, Essays Biochem. (2005)]. Thus, by guiding E3 ubiquitin ligases to a protein of interest, one can mediate its depletion.

Numerous previous works have attempted to redirect E3 ubiquitin ligases by replacing their natural protein binding domains with those targeting specific proteins [Gosink, M. M. et al., “Redirecting the specificity of ubiquitination by modifying ubiquitin-conjugating enzymes”, PNAS (1995); Zhou, et al., “Harnessing the ubiquitination machinery to target the degradation of specific cellular proteins”, Mol. Cell (2000); Su, et al., “Eradication of pathogenic β-catenin by Skpl/Cullin/F box ubiquitination machinery”, PNAS (2003)]. Recently, the TRIM-Away method was devised, wherein antibodies targeting specific proteins would be recognized, via their Fc domain, by the exogenously expressed human TRIM21 E3 ubiquitin ligase, thus facilitating rapid and acute protein degradation [Clift, et al., “Acute and rapid degradation of endogenous proteins by Trim-Away”, Cell (2018)].

In 2014, Portnoff, et al. reprogrammed the substrate specificity of a modular human E3 ubiquitin ligase called CHIP (carboxyl-terminus of Hsc70-interacting protein) by replacing its natural substrate-binding domain with designer binding proteins to generate optimized “ubiquibodies” or uAbs [Portnoff, A. D., Stephens, E. A., Varner, J. D. & DeLisa, M. P., “Ubiquibodies, synthetic e3 ubiquitin ligases endowed with unnatural substrate specificity for targeted protein silencing”, J. Biol. Chem., 289, 7844-7855 (2014)].

Regulatory T cells (Tregs) are a specialized subpopulation of T cells that act to suppress immune response, thereby maintaining homeostasis and self-tolerance [Kwon, et al., “Different molecular complexes that mediate transcriptional induction and repression by FoxP3”, Nat. Immunol. (2018)]. Conversely, the immunosuppressive activity of Tregs may contribute to the progression of cancer or infectious diseases by preventing the induction of specific immune responses [Casares, et al., “A Peptide Inhibitor of FOXP3 Impairs Regulatory T Cell Activity and Improves Vaccine Efficacy in Mice”, J Immunol. (2010)].

FOXP3 is a transcription factor (TF) of the Forkhead family and is specifically expressed in T regulatory cells (Tregs). Its expression is essential for Treg differentiation and function, and is the defining factor of the lineage. FOXP3 loss-of-function leads to Treg deficiency, and thus serves as a druggable target for therapeutic modalities in the tumor microenvironment [Rudensky, “Regulatory T Cells and Foxp3”, Immunol Rev. (2012)].

### SUMMARY

In some aspects, the present invention includes peptide-E3 ubiquitin ligase fusions that represent the most minimal protein to proteasome linkers, specifically targeted to degrade the endogenous FOXP3 protein. These fusions can mediate targeted and robust degradation of exogenously-expressed FOXP3 proteins in T regulatory cells.

In some aspects, the invention is an engineered peptide for functional inactivation of a target regulatory T cell, comprising a fusion protein comprising a targeting domain and a ubiquitin ligase recruiting domain, wherein the targeting domain is engineered to bind FOXP3 of the target regulatory T cell for mediated degradation by the ubiquitin-proteosome pathway. In some embodiments, the targeting domain may comprise a peptide having an amino acid sequence that is RDFQSFRKMWPFFAM [SEQ ID No. 3], RAFQSFRKMWPFFAM [SEQ ID No. 4], RAFQAFRKMWPFFAM [SEQ ID No. 5], LKLRNADIELRKGETDIGRKN [SEQ ID No. 6], or RDFQSFRKMWPFFAM [SEQ ID No. 7], or is at least 90% or 80% identical to those sequences. In some embodiments, the ubiquitin ligase recruiting domain recruits an E3 ubiquitin ligase. The E3 ubiquitin ligase may be CHIPΔR [SEQ ID No. 2].

In other aspects, the present invention is an engineered minimal, specific, nucleotide-encodable, FOXP3 protein to proteasome linker, which may comprise a peptide-E3 ubiquitin ligase fusion in which the peptide binds to FOXP3. In some embodiments, the peptide may have an amino acid sequence that is RDFQSFRKMWPFFAM [SEQ ID No. 3], RAFQSFRKMWPFFAM [SEQ ID No. 4], RAFQAFRKMWPFFAM [SEQ ID No. 5], LKLRNADIELRKGETDIGRKN [SEQ ID No. 6], or RDFQSFRKMWPFFAM [SEQ ID No. 7]], or is at least 90% or 80% identical to those sequences. In some embodiments, the E3 ubiquitin ligase may be CHIPΔR [SEQ ID No. 2].

In another aspect, the invention includes a method for the treatment or alleviation of a medical condition in a subject comprising: administering to the subject an engineered peptide-based therapeutic or pharmaceutically acceptable salt thereof, wherein the engineered peptide-based therapeutic comprises a peptide fusion of a targeting domain and a ubiquitin ligase recruiting domain, and wherein the targeting domain is engineered to bind FOXP3 of at least one regulatory T cell for mediated degradation by the ubiquitin-proteosome pathway. In some embodiments, the targeting domain comprises a peptide having an amino acid sequence is RDFQSFRKMWPFFAM [SEQ ID No. 3], RAFQSFRKMWPFFAM [SEQ ID No. 4], RAFQAFRKMWPFFAM [SEQ ID No. 5], LKLRNADIELRKGETDIGRKN [SEQ ID No. 6], or RDFQSFRKMWPFFAM [SEQ ID No. 7], or is at least 90% or 80% identical to those sequences. In some embodiments, the ubiquitin ligase recruiting domain recruits an E3 ubiquitin ligase, which may be CHIPΔR [SEQ ID No. 2]. The peptide-based therapeutic may be coupled to a delivery vector in which the delivery vector is either a virus or micelle. The peptide fusion may be further fused to a cell penetrating motif or a cell surface receptor binding motif.

### BRIEF DESCRIPTION OF THE DRAWINGS

Other aspects, advantages and novel features of the invention will become more apparent from the following detailed description of the invention when considered in conjunction with the accompanying drawings, wherein:

FIG. 1 depicts an example computational design pipeline for FOXP3 peptide fusions, according to one aspect of the invention.

FIG. 2 is a diagram illustrating an example of mediated degradation of FOXP3, by recruitment of an E3 ubiquitin ligase by a fusion protein for an example FOXP3 peptide fusion, according to an embodiment of the present invention.

FIG. 3 is a graph depicting experimental results for FOXP3 degradation via example peptide fusions, demonstrating the efficacy of the example mediated degradation of FIG. 2.

### DETAILED DESCRIPTION

The present invention includes peptide-E3 ubiquitin ligase fusions that represent the most minimal protein to proteasome linkers, specifically targeted to degrade the endogenous FOXP3 protein in regulatory T cells. The underlying methodology employed herein is discussed in additional detail in Chatterjee, P., Ponnapati, M., Kramme, C., Plesa, A.M., Church, G. M. and Jacobson, J. M., “Targeted intracellular degradation of SARS-COV-2 via computationally optimized peptide fusions”, Communications biology, 3 (1), pp. 1-8 (2020) and U.S. patent application Ser. No. 17/334,055, filed May 28, 2021, which are hereby incorporated by reference herein in their entirety.

The invention facilitates mediated degradation of regulatory T cells by using fusion peptides for targeting the endogenous FOXP3 protein of regulatory T cells for intracellular degradation. The invention leverages computational design of fusion peptides for the mediation of regulatory T cell degradation. In particular, the invention encompasses engineered fusion peptides for targeting FOXP3 for mediation degradation by the ubiquitin-proteosome pathway. It includes peptide-E3 ubiquitin ligase fusions that represent a minimal protein to proteasome linker. These fusion peptides can mediate targeted and robust degradation of expressed intracellular proteins with expression of a cognate E3 ubiquitin ligase. In certain preferred embodiments, the target-binding peptide is fused directly to any E3 Ubiquitin Ligase such as, but not limited to, CHIPΔR or TRIM 21.

### Computational Peptide Design

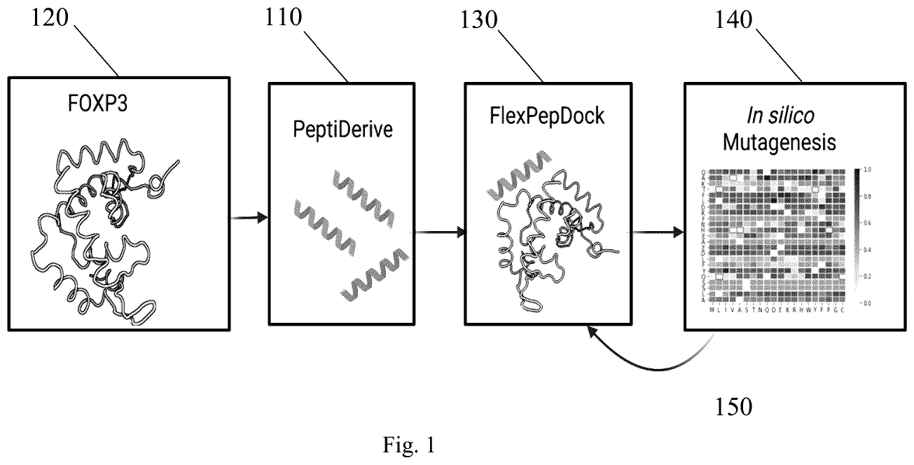

FIG. 1 depicts a computational design pipeline for FOXP3 peptides. FOXP3 was isolated from PDB 4WK8. The canonical FOXP3 amino acid sequence is:

[SEQ ID No. 1]
MPNPRPGKPSAPSLALGPSPGASPSWRAAPKASDLLGARGPGGTF
QGRDLRGGAHASSSSLNPMPPSQLQLPTLPLVMVAPSGARLGPLP
HLQALLQDRPHFMHQLSTVDAHARTPVLQVHPLESPAMISLTPPT
TATGVFSLKARPGLPPGINVASLEWVSREPALLCTFPNPSAPRKD
STLSAVPQSSYPLLANGVCKWPGCEKVFEEPEDFLKHCQADHLLD
EKGRAQCLLQREMVQSLEQQLVLEKEKLSAMQAHLAGKMALTKAS
SVASSDKGSCCIVAAGSQGPVVPAWSGPREAPDSLFAVRRHLWGS
HGNSTFPEFLHNMDYFKFHNMRPPFTYATLIRWAILEAPEKQRTL
NEIYHWFTRMFAFFRNHPATWKNAIRHNLSLHKCFVRVESEKGAV
WTVDELEFRKKRSQRPSRCSNPTPGP

Two FOXP3 amino acid chains were designated as Chain A and Chain B. As shown in FIG. 1, the PeptiDerive protocol 110 was applied to FOXP3 120 to produce candidate peptide binders. Candidate binders were docked in silico utilizing FlexPepDock 130 to downselect candidate peptides, which were then subjected to mutational scans 140 and redocking 150 with FlexPepDock 130 to optimize binding properties.

PDB Structure 4WK8 containing the crystal structure of FOXP3 bound to its DNA partner sequence was retrieved. The PeptiDerive protocol in the Rosetta protein modeling software [Sedan, et al., “Peptiderive server: derive peptide inhibitors from protein-protein interactions”, Nucleic Acids Research (2016)] was used to determine the linear peptide segments between 10 and 23 amino acids of each of the two chains of FOXP3 with significant binding energy to the partner chain. To analyze the conformational entropy of the peptide segments in the binding pocket, a combination of FlexPepDock [Raveh, et al., “Rosetta FlexPepDock ab-initio: Simultaneous Folding, Docking and Refinement of Peptides onto Their Receptors”, PLOS ONE (2011)] and protein-protein docking protocols in Rosetta was used to dock the peptides to the original FOXP3 protein after removal of DNA substrate. Using FlexPepDock, 300 models were created for each peptide and the top 15 models were selected to calculate the score.

High-throughput in silico truncations to the FOXP3 heterodimer were conducted via the Peptiderive and FlexPepDock servers [Sedan, et al., “Peptiderive server: derive peptide inhibitors from protein-protein interactions”, Nucleic Acids Research (2016); Raveh, et al., “Rosetta FlexPepDock ab-initio: Simultaneous Folding, Docking and Refinement of Peptides onto Their Receptors”, PLOS ONE (2011)]. This minimized the FOXP3 protein structure to the sufficient components needed for binding to the alternate chain of FOXP3 (FIG. 1). The PeptiDerive algorithm was applied multiple times for each peptide length between 10 and 23 amino acids to find candidates derived from each FOXP3 amino acid chain which binds to the paired chain with high affinity. Each candidate protein was computationally relaxed, and those with the lowest total energy score, and thus highest binding affinity, were selected for experimental analysis.

The candidate peptide sequences were fused via a short, flexible linker of GSGSG to the 5′ end of CHIPΔR, an optimized human-derived E3 ubiquitin ligase, as described by Portnoff, et al. [Portnoff, et al., “Ubiquibodies, Synthetic E3 Ubiquitin Ligases Endowed with Unnatural Substrate Specificity for Targeted Protein Silencing”, J Biol Chem. (2014)] and having the sequence:

[SEQ ID No. 2]
MRLNFGDDIPSALRIAKKKRWNSIEERRIHQESELHSYLSRLIAA
ERERELEECQRNHEGDEDDSHVRAQQACIEAKHDKYMADMDELFS
QVDEKRKKRDIPDYLCGKISFELMREPCITPSGITYDRKDIEEHL
QRVGHFDPVTRSPLTQEQLIPNLAMKEVIDAFISENGWVEDY

While the example embodiments employ CHIPΔR, it will be clear to one of skill in the art that engineered variants of CHIPΔR, and other human E3 ligases capable of catalyzing the transfer of ubiquitin to target proteins such as, but not limited to, TRIM21, will be suitable for use in the present invention.

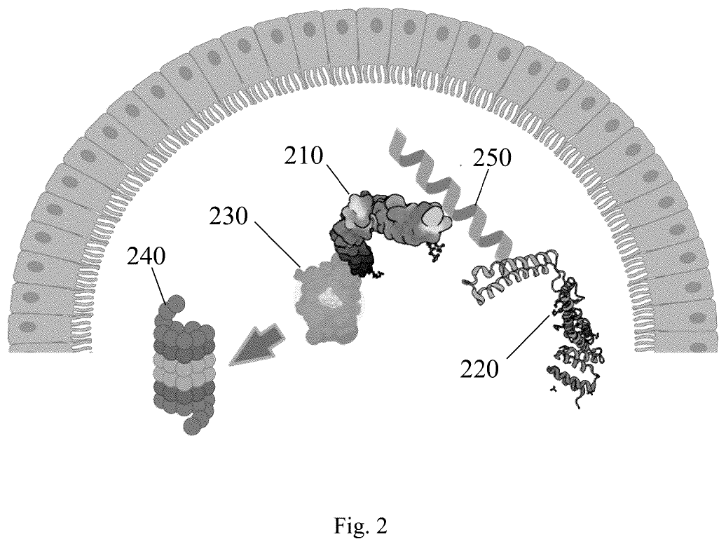

FIG. 2 is a diagram illustrating an experimental pipeline for testing FOXP3 peptide fusions. In FIG. 2, an example of mediated degradation of FOXP3 210, peptides 250 are fused to E3 ubiquitin ligase 220, in this case CHIPΔR, and co-transfected into HEK293T cells with a FOXP3 210-sfGFP 230 reporter fusion. Peptide 250 binding to exogenous FOXP3 210 induces degradation and loss of sfGFP (stable superfolder-green fluorescent protein) 230 signal through the action of proteasome 240. In this particular example, FOXP3 210 is bound by targeting domain P60 250 of the fusion protein, which protein comprises the targeting domain 250 fused directly to the E3 ubiquitin ligase CHIPΔR 220.

Similar fusions were conducted with FOXP3-targeting peptides P60-S2A, and P60-D2A-S5A, identified previously through experimental screening assays [Lozano, et al., “Blockage of FOXP3 transcription factor dimerization and FOXP3/AML1 interaction inhibits T regulatory cell activity: sequence optimization of a peptide inhibitor”, Oncotarget (2017)].

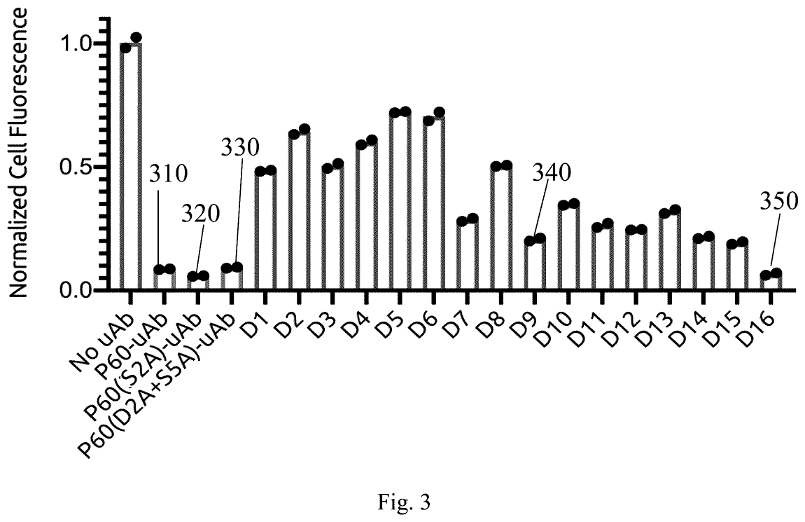

For testing, for example in human HEK293T cells, the FOXP3 protein was fused to sfGFP, expressed, and tested for sfGFP degradation. FIG. 3 is a graph depicting experimental results for FOXP3 degradation via peptide fusions. 19 different peptide designs were fused to CHIPΔR and individually co-transfected into HEK293T cells with a FOXP3-sfGFP reporter fusion. Resulting degradation is measured as the normalized fluorescence as compared to an undegraded control. Samples were performed in individual biological duplicates (n=2).

The candidates shown experimentally to be most successful 310, 320, 330, 340, 350 (FIG. 3) are shown in Table 1. While these candidates were identified experimentally as being particularly successful, it will be clear to one of skill in the art of the invention that the usage of any peptide sequence, where a peptide is defined as any amino acid sequence shorter than 200 amino acids, in conjunction with sequences that are already existent in nature/literature, is within the scope of the present invention. Therefore, example peptide sequences within the scope of the invention include, but are not limited to the sequences listed in Table 1.

TABLE 1
P60: RDFQSFRKMWPFFAM
[SEQ ID No. 3] (310)
P60-S2A: RAFQSFRKMWPFFAM
[SEQ ID No. 4] (320)
P60-D2A-S5A: RAFQAFRKMWPFFAM
[SEQ ID No. 5] (330)
D9: LKLRNADIELRKGETDIGRKN
[SEQ ID No. 6] (340)
D16: RDFQSFRKMWPFFAM
[SEQ ID No. 7] (350)

Other targeting domain peptide sequences may include, but are not limited to, sequences derived by the methods disclosed in U.S. patent application Ser. No. 17/222,676, the entirety of which is incorporated herein by reference.

This reduction to practice demonstrates that these fusions can mediate targeted and robust degradation of exogenously expressed FOXP3 proteins. In particular, the results demonstrate that a subset of the tested peptide fusions (the P60 variants 310, 320, 330 and D9 340 and D16 350) were capable of mediating robust degradation of FOXP3-sfGFP (superfolder green fluorescent protein) fusion proteins within HEK239T cells (FIG. 1).

While the invention has been illustrated herein by embodiments employing these specific example peptides, it will be clear to one of skill in the art that variants of these peptides that are similar in amino acid sequence, and other, related peptides having similar properties, will also be suitable for use in the present invention. Thus, amino acid sequences possessing sequence homology of greater than 80% to the example embodiments of peptide-E3 ubiquitin ligase fusions in which the peptide binds to FOXP3 that are disclosed herein are considered to be within the scope of the invention.

The peptide-E3 ubiquitin ligase fusions specifically targeted to degrade the FOXP3 protein can be employed as therapeutics. Among those specifically contemplated are a peptide-based therapeutic comprising a peptide-E3 ubiquitin ligase fusion in which the peptide has amino acid sequence RAFQSFRKMWPFFAM [SEQ ID No. 4] and a peptide-E3 ubiquitin ligase fusion in which the peptide has amino acid sequence RAFQAFRKMWPFFAM [SEQ ID No. 5], coupled to a delivery vector which is either a virus or micelle by techniques known in the art. Also contemplated are a peptide-based therapeutic comprising the a peptide-E3 ubiquitin ligase fusion in which the peptide binds to FOXP3, in which the peptide fusion is further fused to a cell penetrating motif or a cell surface receptor binding motif by techniques known in the art.

The platform of the present invention provides a rapid and direct targeting mechanism, which coupled with its size and human-protein derivation, presents numerous advantages as compared with existing strategies. The strategy of utilizing a computationally-designed peptide binder linked to an E3 ubiquitin ligase can be effective not only for FOXP3 of regulatory T-cells, but also for other drug targets and for viruses that have known binding partners. With already over 30,000 co-crystal structures currently in the PDB, and structure determination becoming more routine with advances in cryogenic electron microscopy, the computational peptide engineering pipeline presented here provides a versatile new therapeutic platform in the fight against numerous diseases.

In one aspect, the invention includes a method of using any short peptide (such as the examples listed in Table 1), fused to any E3 Ubiquitin ligase, to degrade a protein of interest in a human cell. For peptides fused to Fc, an E3 Ubiquitin ligase that can be co-expressed is TRIM 21. The peptide can be directly fused to any E3 Ubiquitin ligase [Medvar B, Raghuram V, Pisitkun T, Sarkar A, Knepper M A, “Comprehensive database of human E3 ubiquitin ligases: application to aquaporin-2 regulation. Physiol Genomics. 2016; 48 (7): 502-512]. In a preferred embodiment, an E3 Ubiquitin ligase that can be used is CHIPΔR.

In another application, a method according to the invention is used for the treatment or alleviation of a medical condition in a subject. The method includes administering an engineered peptide-based therapeutic, or pharmaceutically acceptable salt thereof, to the subject. The engineered peptide-based therapeutic comprises a peptide fusion of a targeting domain and a ubiquitin ligase recruiting domain, wherein the targeting domain is engineered to bind FOXP3 of at least one regulatory T cell for mediated degradation by the ubiquitin-proteosome pathway. In some embodiments, the targeting domain comprises a peptide having the amino acid sequence RDFQSFRKMWPFFAM [SEQ ID No. 3], RAFQSFRKMWPFFAM [SEQ ID No. 4], RAFQAFRKMWPFFAM [SEQ ID No. 5], LKLRNADIELRKGETDIGRKN [SEQ ID No. 6], or RDFQSFRKMWPFFAM [SEQ ID No. 7], or is at least 90% or 80% identical to those sequences. In some embodiments, the ubiquitin ligase recruiting domain recruits an E3 ubiquitin ligase. In a preferred embodiment, the E3 ubiquitin ligase employed is CHIPΔR [SEQ ID No. 2].

### Generation of Plasmids

pcDNA3-SARS-COV-2-S-RBD-sfGFP (Addgene #141184) and pcDNA3-R4-uAb (Addgene #101800) were obtained as gifts. hFOXP3 was synthesized as a gBlock from Integrated DNA Technologies (IDT), PCR amplified, and inserted into the lincarized backbone of pcDNA3-SARS-COV-2-S-RBD-sfGFP following digestion with Nhel and BamHI. Respective peptide DNA coding sequences (CDS) were inserted via site-directed mutagenesis using the KLD Enzyme Mix (NEB) into pcDNA3-R4-uAb post digestion with HindIII and EcoRI. Assembled constructs were transformed into 50 μL. NEB Turbo Competent Escherichia coli cells, and plated onto LB agar supplemented with the appropriate antibiotic for subsequent sequence verification of colonies (Genewiz) and plasmid purification.

### Cell Culture and Flow Cytometry Analysis

HEK293T cells were maintained in Dulbecco's Modified Eagle's Medium supplemented with 100 units/ml penicillin, 100 mg/ml streptomycin, and 10% fetal bovine serum (FBS). hFOXP3-sfGFP (250 ng) and peptide-CHIPΔR (250 ng) plasmids were transfected into cells as duplicates (2×10⁵/well in a 24-well plate) with Lipofectamine 3000 (Invitrogen) in Opti-MEM (Gibco). After 3 days post transfection, cells were harvested and analyzed on an FACS Celesta (BD) for GFP fluorescence (488-nm laser excitation, 530/30 filter for detection). Cells expressing GFP were gated, and percent GFP+ to the FOXP3-sfGFP only control were calculated for normalization. All samples were performed in independent transfection duplicates (n=2), and percentage depletion values were averaged.

At least the following aspects, implementations, modifications, and applications of the described technology are contemplated by the inventors and are considered to be aspects of the presently claimed invention: (1) A minimal, specific, nucleotide encodable, FOXP3 protein to proteasome linker; (2) The linker of (1), comprising a peptide-E3 ubiquitin ligase fusion in which the peptide binds to FOXP3; (3) The linker of (2), comprising a peptide-E3 ubiquitin ligase fusion in which the peptide has amino acid sequence: RDFQSFRKMWPFFAM [SEQ ID No. 3]; (4) The linker of (2), comprising a peptide-E3 ubiquitin ligase fusion in which the peptide has amino acid sequence: RAFQSFRKMWPFFAM [SEQ ID No. 4]; (5) The linker of (2), comprising a peptide-E3 ubiquitin ligase fusion in which the peptide has amino acid sequence: RAFQAFRKMWPFFAM [SEQ ID No. 5]; (6) The linker of (2), comprising a peptide-E3 ubiquitin ligase fusion in which the peptide is of amino acid sequence: LKLRNADIELRKGETDIGRKN [SEQ ID No. 6]; (7) The linker of (2), comprising a peptide-E3 ubiquitin ligase fusion in which the peptide is of amino acid sequence: RDFQSFRKMWPFFAM [SEQ ID No. 7]; (8) The linker of (2), (3), (4), (5), (6), or (7), comprising a peptide-E3 ubiquitin ligase fusion in which the E3 ubiquitin ligase is of amino acid sequence:

[SEQ ID No. 2]
MRLNFGDDIPSALRIAKKKRWNSIEERRIHQESELHSYLSRLIAA
ERERELEECQRNHEGDEDDSHVRAQQACIEAKHDKYMADMDELFS
QVDEKRKKRDIPDYLCGKISFELMREPCITPSGITYDRKDIEEHL
QRVGHFDPVTRSPLTQEQLIPNLAMKEVIDAFISENGWVEDY;

(9) Amino acid sequences possessing sequence homology of greater than 80% to the linkers of (2), (3), (4), (5), (6), (7), or (8); (10) Peptide-based therapeutic comprising the polynucleotide of (4) and (5) coupled to a delivery vector in which the delivery vector may be either a virus or micelle; and (11) Peptide-based therapeutic comprising the fusions of (2) and (3), in which the peptide fusion is further fused to a cell penetrating motif or a cell surface receptor binding motif.

While preferred embodiments of the invention are disclosed herein, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

What is claimed is:

1. An engineered peptide for functional inactivation of a target regulatory T cell, comprising a fusion protein comprising a targeting domain and an E3 ubiquitin ligase, wherein the targeting domain is engineered to bind FOXP3 of the target regulatory T cell for mediated degradation by the ubiquitin-proteosome pathway and comprises a peptide having an amino acid sequence selected from the group consisting of RAFQSFRKMWPFFAM [SEQ ID No. 4], RAFQAFRKMWPFFAM [SEQ ID No. 5], and LKLRNADIELRKGETDIGRKN [SEQ ID No. 6], and wherein the E3 ubiquitin ligase is CHIPΔTPR, comprising the amino acid sequence:
[SEQ ID No. 2]
MRLNFGDDIPSALRIAKKKRWNSIEERRIHQESELHSYLSRLIAA
ERERELEECQRNHEGDEDDSHVRAQQACIEAKHDKYMADMDELFS
QVDEKRKKRDIPDYLCGKISFELMREPCITPSGITYDRKDIEEHL
QRVGHFDPVTRSPLTQEQLIPNLAMKEVIDAFISENGWVEDY.

2. The engineered peptide of claim 1, wherein the engineered peptide is coupled to a delivery vector in which the delivery vector is either a virus or a micelle.

3. The engineered peptide of claim 1, wherein the engineered peptide is further fused to a cell penetrating motif or a cell surface receptor binding motif.

4. A method for the treatment or alleviation of a medical condition in a subject comprising: administering to the subject an engineered peptide-based therapeutic or pharmaceutically acceptable salt thereof, wherein the engineered peptide-based therapeutic comprises the engineered peptide of claim 1.

5. The method of claim 4, wherein the peptide-based therapeutic is coupled to a delivery vector in which the delivery vector is either a virus or micelle.

6. The method of claim 4, wherein the engineered peptide is further fused to a cell penetrating motif or a cell surface receptor binding motif.

7. An engineered peptide for functional inactivation of a target regulatory T cell, comprising a fusion protein comprising a targeting domain and an E3 ubiquitin ligase, wherein the targeting domain is engineered to bind FOXP3 of the target regulatory T cell for mediated degradation by the ubiquitin-proteosome pathway and comprises a peptide having an amino acid sequence with at least 90% amino acid sequence identity to a sequence selected from the group consisting of RAFQSFRKMWPFFAM [SEQ ID No. 4], RAFQAFRKMWPFFAM [SEQ ID No. 5], and LKLRNADIELRKGETDIGRKN [SEQ ID No. 6], and wherein the E3 ubiquitin ligase is CHIPΔR, comprising the amino acid sequence:
[SEQ ID No. 2]
MRLNFGDDIPSALRIAKKKRWNSIEERRIHQESELHSYLSRLIAA
ERERELEECQRNHEGDEDDSHVRAQQACIEAKHDKYMADMDELFS
QVDEKRKKRDIPDYLCGKISFELMREPCITPSGITYDRKDIEEHL
QRVGHFDPVTRSPLTQEQLIPNLAMKEVIDAFISENGWVEDY.

8. The engineered peptide of claim 7, wherein the engineered peptide is coupled to a delivery vector in which the delivery vector is either a virus or a micelle.

9. The engineered peptide of claim 7, wherein the engineered peptide is further fused to a cell penetrating motif or a cell surface receptor binding motif.

10. A method for the treatment or alleviation of a medical condition in a subject comprising: administering to the subject an engineered peptide-based therapeutic or pharmaceutically acceptable salt thereof, wherein the engineered peptide-based therapeutic comprises the engineered peptide of claim 7.

11. The method of claim 10, wherein the peptide-based therapeutic is coupled to a delivery vector in which the delivery vector is either a virus or micelle.

12. The method of claim 10, wherein the engineered peptide is further fused to a cell penetrating motif or a cell surface receptor binding motif.

13. An engineered peptide for functional inactivation of a target regulatory T cell, comprising a fusion protein comprising a targeting domain and an E3 ubiquitin ligase, wherein the targeting domain is engineered to bind FOXP3 of the target regulatory T cell for mediated degradation by the ubiquitin-proteosome pathway and comprises a peptide having an amino acid sequence with at least 80% amino acid sequence identity to a sequence selected from the group consisting of RAFQSFRKMWPFFAM [SEQ ID No. 4], RAFQAFRKMWPFFAM [SEQ ID No. 5], and LKLRNADIELRKGETDIGRKN [SEQ ID No. 6], and wherein the E3 ubiquitin ligase is CHIPΔR, comprising the amino acid sequence:
[SEQ ID No. 2]
MRLNFGDDIPSALRIAKKKRWNSIEERRIHQESELHSYLSRLIAA
ERERELEECQRNHEGDEDDSHVRAQQACIEAKHDKYMADMDELFS
QVDEKRKKRDIPDYLCGKISFELMREPCITPSGITYDRKDIEEHL
QRVGHFDPVTRSPLTQEQLIPNLAMKEVIDAFISENGWVEDY.

14. The engineered peptide of claim 13, wherein the engineered peptide is coupled to a delivery vector in which the delivery vector is either a virus or a micelle.

15. The engineered peptide of claim 13, wherein the engineered peptide is further fused to a cell penetrating motif or a cell surface receptor binding motif.

16. A method for the treatment or alleviation of a medical condition in a subject comprising: administering to the subject an engineered peptide-based therapeutic or pharmaceutically acceptable salt thereof, wherein the engineered peptide-based therapeutic comprises the engineered peptide of claim 9.

17. The method of claim 16, wherein the peptide-based therapeutic is coupled to a delivery vector in which the delivery vector is either a virus or micelle.

18. The method of claim 16, wherein the engineered peptide is further fused to a cell penetrating motif or a cell surface receptor binding motif.


SEQUENCE LISTING (7 sequences)

SEQ ID NO: 1 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 2 - protein, 177 aa, Homo sapiens
MRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 3 - protein, 15 aa, synthetic construct
RDFQSFRKMWPFFAM

SEQ ID NO: 4 - protein, 15 aa, synthetic construct
RAFQSFRKMWPFFAM

SEQ ID NO: 5 - protein, 15 aa, synthetic construct
RAFQAFRKMWPFFAM

SEQ ID NO: 6 - protein, 21 aa, synthetic construct
LKLRNADIELRKGETDIGRKN

SEQ ID NO: 7 - protein, 15 aa, synthetic construct
RDFQSFRKMWPFFAM